(19) United States
(12) Patent Application Publication
(10) Pub. No.: US 2016/0331993 A1
(43) Pub. Date: Nov. 17, 2016

(54) LIGHT EMITTING DIODE BASED SKIN TREATMENT DEVICE FOR THE TREATMENT OF DERMATOLOGIC CONDITIONS AND METHOD OF USE

(71) Applicant: Hunter Reid Moyer, Cumming, GA (US)

(72) Inventor: Hunter Reid Moyer, Cumming, GA (US)

(21) Appl. No.: 14/711,831

(22) Filed: May 14, 2015

(51) Int. Cl. A61N 5/06

(52) U.S. Cl. CPC A61N 5/0616; A61N 2005/0629; A61N 2005/0652; A61N 2005/0662; A61N 2005/0661

(57) ABSTRACT

This invention is directed to an LED based, skin treatment device designed to physically or wirelessly attach to and be run by an application from a smart device, such as a smart phone or tablet. The treatment device includes a light-emitting diode (LED) array of one or more specific wavelengths within the ultraviolet-visible light range of the electromagnetic spectrum. The treatment device may attaches to the working port of a smartphone and powered and controlled by an application or may alternatively be run by the same application via a wireless connection.

### BACKGROUND OF THE INVENTION

Human skin is exposed to a multitude of environmental stressors throughout daily life. The inherent production of collagen, elastin and melanin decreases as we age leaving skin with wrinkles and heterochromias. Several ailments—including (but not exclusive of) acne, rosacea, vitiligo and skin aging—are effectively treated by blue, red and ultraviolet LED lights. In addition, new research has shown an alleviating effect of LED therapy for osteopathic pain and neuropathic pain.

Known methods to reduce or smooth wrinkles include resurfacing procedures, botulinum toxin injection, and the injection of subcutaneous and/or dermal fillers. Results from these procedures are temporary, and each therapy is invasive. Recently, the application of red LED light has been proven to increase in collagen production, reduce fine lines and wrinkles, and decrease the size and color of sunspots. Red light, with a central wavelength of 635 nm, increases ATP production within a cell thereby improving cell turnover and increasing cell oxygenation. Narrowband red light from an LED has shown the most promising results.

Acne vulgaris is a common, debilitating skin ailment defined by inflammation and infection in the oil glands of the skin. The result is painful pustules and papules that can lead to scarring. Proprionibacterium acnes is an anaerobic bacterium that is known to contribute to the development of pimples and pustules. An established method to prevent and treat active acne is the serial application of a blue LED light at 40 mW/cm². The light, with a centered wavelength of 465 nm, is selectively absorbed by porphyrins in the bacteria and once absorbed causes the death of the bacteria via free radical production. Recently, practioners have added red LED light therapy (635 nm) in combination with blue light to enhance the efficacy.

Rosacea is a chronic skin malady defined by skin redness, superficial blood vessels, swelling and papules and pustules. The ailment is painful and can cause social stress and disfiguring scars. Treatments include behavioral changes, the topical application of acids, antibiotics, and beta-blockers as well as LASER therapy. Recently, red LED light (635 nm) and infrared LED light (695 nm) have been used to treat the flushing and inflammation common in rosacea. Results are promising, but the treatments need to be delivered frequently; thus, a small and readily available device for administration “on-the-go” is needed.

Vitiligo is a skin condition defined by loss of pigmentation. The cause is unknown, but most research suggests autoimmune, genetic, viral or oxidative stress mechanisms. The ailment is not painful, but affected individuals often suffer stigmata leading to depression and mood disorders. Treatments are limited. The best, modern therapy combines topical steroids and ultraviolet phototherapy. Narrowband UVB light (wavelength of 315 nm) and UVA light combined with psoralen is the most effective treatment for re-pigmentation. Treatments need to be delivered daily, and therefore accessibility is generally the most limiting factor for cure.

LED light therapy is likely a promising technology for the treatment of other skin maladies as yet undefined or untreated. No matter the condition, it is readily apparent that treatments must be administered frequently. Thus, the medical community needs an inexpensive device that can be placed in the hands of the patient to ease the burden of the treatment protocol. Many home devices have been developed; however, with the boom in smartphone utilization and ease of creating a controlling app, this patent applies to the use of a smartphone and peripheral for delivery of light therapies.

### BRIEF SUMMARY OF THE INVENTION

Embodiments of the invention may include an LED based, skin treatment peripheral device comprising an LED array having at least one LED, where the at least one LED emits a wavelength within the ultraviolet and visible light range of electromagnetic spectrum. The skin treatment peripheral device also includes an LED control module adapted to receive instructions or data from a smart device and turn the at least one LED in the LED array on and off for predetermined periods of time based on the instructions received from the smart device.

Still further, embodiments of the invention may include a method for treating a skin condition comprising the steps of providing at least one skin treatment protocol within an application on a smart device, where the at least one skin treatment protocol provides a predetermined wavelength of light for a predetermined period of time. The method includes connecting an LED based, skin treatment peripheral device to the smart device, where the LED based, skin treatment apparatus comprises a LED array comprising at least one LED, where the at least one LED emits a wavelength corresponding to the predetermined wavelength of light from the protocol, and an LED control module adapted to receive instructions from the smart device and turn the at least one LED in the LED array on and off for the predetermined periods of time based on the treatment protocol. The method also includes the step of selecting a skin treatment protocol from the at least one skin treatment protocol, running the selected skin treatment protocol and exposing the skin of a patient to the LED based, skin treatment peripheral device during the step of running the selected skin treatment protocol.

### BRIEF DESCRIPTION OF THE DRAWING

FIG. 1 is a diagrammatic view showing a peripheral device in accordance with an embodiment of the invention and a smartphone,

### DETAILED DESCRIPTION OF EMBODIMENTS OF THE INVENTION

The present invention provides a small, portable, LED based, skin treatment peripheral device that provides patients with the ability to provide the desired skin treatment virtually anywhere. As many of the skin treatment protocols can extend for several weeks or months, this feature increases the ability of a patient to maintain and comply with a desired skin condition treatment protocol.

As will be discussed in detail below, embodiments of the invention include an LED based, skin treatment peripheral device that can be controlled directly by an application running on a smart device such as a smart phone or tablet. The LED based, skin treatment peripheral device turns an array of LEDs on and off for predetermined periods of time based on signals received from the smart device corresponding to selected skin treatment protocols. While smart devices may include, but is not limited to, smart phones, smart watches, tablets, laptop computers, tablet computers, desk top computers, and other similar electronic devices, embodiments of the invention will be described below in relation to a smart phone. One skilled in the art will understand and be able to readily apply the features of this invention with a wide range of smart devices.

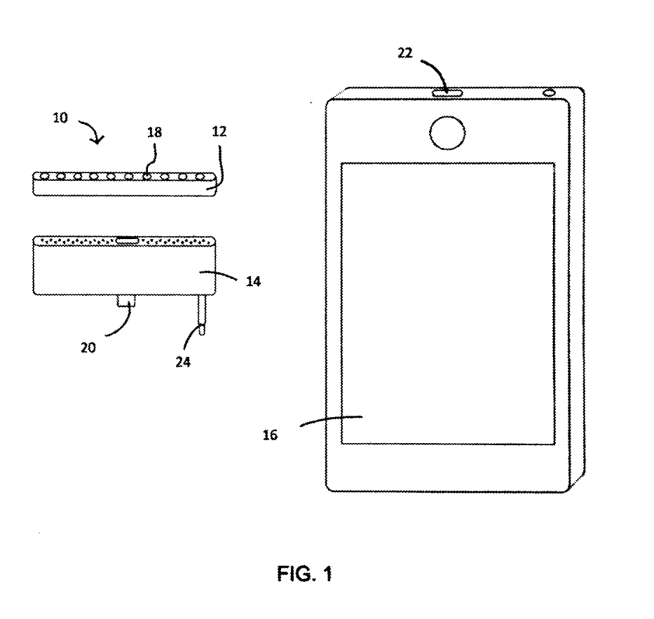

With reference now to FIG. 1, there is shown an LED based skin treatment peripheral device in accordance with an embodiment of the invention designated generally by the reference numeral 10. As used herein, “LED” refers to light-emitting diode. In general, the LED based skin treatment peripheral device 10 comprises an LED array 12 and an LED controller module 14. The LED controller module 14 receives instructions from a smart device 16 such as a smart phone or tablet to control the LED array 12.

The LED array comprises one or more LEDs. One such LED being designated by the reference numeral 18. The position or configuration of the LEDs 18 in the LED array 12 is not particularly limited and may take on one or more linear rows, either straight or curved. Alternatively the LEDs may be positioned in one or more circles or other geometric configurations. The LEDs preferably exhibit a peak or central wavelength in the range from about 200 nm to about 800 nm. In some embodiments, the LEDs preferably provide an intensity of about 40 mW/cm². The LEDs in the LED array may exhibit the same central wavelength while in other embodiments, at least two different LEDs in the LED array exhibit different central wavelengths. In some embodiments, one or more of the LEDs in the LED array exhibits a central wavelength of about 695 nm, about 635 nm, about 465 nm, or about 315 nm. The LED array is preferably encased in a durable polymer or plastic material compatible with the LEDs in the LED array.

The LED array 12 may be removable or otherwise detachable from the LED controller module 14. The LED array 12 and the LED controller module 14 may be connected together via a series of pins between the LED array and the LED controller module or other suitable connection known to those skilled in the art. In this way, different LED arrays having a different selection of LEDs may be interchanged depending upon the particular treatment protocol. An LED array 12 may include one or more LEDs 18 exhibiting the same wavelength. In other embodiments, the LED array 12 may include at least two LEDs 18 that exhibit different wavelengths. Accordingly, the LED array can encompass a series of LEDs of the same color or varied colors, and the LED controller module can attach to LED arrays of varying monochromatic color schemes or varied color schemes.

In other embodiments the LED array 12 is not removable and may be integrally formed with the LED controller module 14 to form a one piece unit. With a non-removable LED array, different treatment peripheral devices would be needed with different LEDs corresponding to the desired wavelengths for a given treatment protocol.

The LED controller module 14 is adapted to receive instructions or data from the smart phone 16 and turn one or more of LEDs 18 in the LED array 12 on and off for predetermined periods of time based on the instructions received from the smart phone 16, In some embodiments, the LED controller module 14 includes a communication port 20 adapted to physically connect to a data port 22 of a smart device. The communication port 20 may connect directly into the data port of the smart phone by using a connector that is compatible with the particular smart phone, such as for example, the lightening port of the Apple iPhone, or a micro USB port on other smartphone. For the iPhone example, the +data and −data connection pins from the lightening to USB connection are used to transmit instructions and data between the peripheral treatment device and the smart phone. In further embodiments the LED controller module may include a 35 mm pin or “headphone jack” 24 compatible with the particular smart phone for communication between the smart phone and the peripheral treatment device. In still additional embodiments, the LED controller module 14 may include a wireless transducer/receiver such as, for example, an RN41 BlueTooth SMD module to communicate with the smart device. In other embodiments the LED controller module 14 may include a wireless module for typical wireless communication protocols, including but not limited to 802.11a, 802.11g, 802.11n, 802.11ac, and other similar wireless communication protocols. The LED controller module 14 may be encased in a durable polymer or plastic material compatible with the LED controller module. In embodiments where the LED controller module includes both physical connection ports as well as a wireless communication module, the peripheral treatment device may communicate with the smart phone directly, or wirelessly.

Power may be supplied to the LED controller module 14 by incorporating a rechargeable battery within the LED controller module. The rechargeable battery provides power to operate the LED controller module 14 and LED array 12. The rechargeable battery can be charged while connected to the smart phone or in some embodiments, via the smart phone charger by a matching data port included in the LED controller module. Alternatively, in other embodiments, when the LED controller module is physically connected to the smart phone through the communication port, the LED controller may draw the required power directly from the smartphone.

The LED controller module 14 also includes a microcontroller adapted to receive instructions from the smart phone to turn specific LEDs in the LED array on and off for specific periods of time. In some embodiments, the microcontroller may control each individual LED 18 in the LED array 12 separately. In additional embodiments, the microcontroller may control two or more LEDs 18 in the LED array 12 in any variety of combinations with one another. For example, in a peripheral treatment device having an LED array that includes LEDs having two different wavelengths, the LED controller module may selectively turn on the LEDs of one wavelength while keeping the LEDs of the second wavelength turned off. Alternatively, LED controller module may turn on both LEDs with different wavelengths simultaneously.

As discussed above, the LED based skin treatment peripheral device 10 receives instructions for controlling the LEDs 18 in the LED array 12 from a smart phone 16. Preferably, an application that controls the LED based skin treatment peripheral device 10 operationally resides within the memory of the smart phone 16 and provides the user a variety of options for controlling the LED based skin treatment peripheral device 10. In some embodiments, the user may manually control parameters of the LED based skin treatment peripheral device 10 from the smart phone 16. Some of the parameters may include, hut are not limited to, particular LED selection, LED on time, LED off time, LED intensity.

The application may also provide the user with a selection of predetermined treatment protocols based on the skin condition to be treated. While many of the protocols may involve treating skin ailments, protocols may also include treatment for wounds and pain. The predetermined treatment protocols provide specific parameters that correspond to the treatment of the specified skin condition. These protocols in the application may be updated from time to time to provide the user with the most up to date treatment LED treatment protocols. Once the user selects the desired treatment protocol, the application sends appropriate communication signals corresponding to instructions in the selected treatment protocol to the LED based skin treatment peripheral device 10 through either the connection port 22 or through the wireless connection (not shown). The LED control module 14 processes these signals and activates the LEDs 18 in the LED array 12 in accordance with the signals and instructions received from the smart phone 16.

Importantly, the LED based skin treatment peripheral device 10 should contain LEDs having the required wavelength and intensity corresponding to the selected treatment protocol. In embodiments with removable LED arrays, an LED array comprising LEDs with the proper wavelengths may be attached to the LED control module. In embodiments with fixed LED arrays, the appropriate LED based skin treatment peripheral device comprising the proper wavelengths is connected to the smart phone.

In certain embodiments where the smart phone or device includes a camera, the application can advantageously utilize the camera functionality to capture images of the treated skin and create a photo log corresponding to the treatment. The photo documentation of results may in some cases act as a platform for improving compliance of therapy. Further, the application can provide for communication with a care provider. The application can send treatment information and the photo log to a health care provider. In some embodiments, the application may facility communication with other users to share or compare information.

In use, the user or patient may select a skin treatment protocol from a list of options provided from the smart phone application. The LED based skin treatment peripheral device having the correct combination of LEDs for the selected skin treatment protocol is connected to the smart phone either physically or wireless to establish communication with the smart phone application. The user or patient runs the selected skin treatment protocol from the application on the smart phone and exposes the skin to be treated to the LEDs of the LED array. The LEDs should be positioned in close proximity to the skin to provide a therapeutic treatment benefit.

In some embodiments the LED based skin treatment peripheral device may be driven in a similar method as described above from a web based application from a computer.

While the present invention has been described in detail with respect to certain embodiments, one skilled in the art will understand the application and broad utility of the present invention. The present invention is to be limited only by the appended claims.

What is claimed is:

1. An LED based, skin treatment peripheral device, the peripheral device comprising:
an LED array comprising at least one LED, wherein the at least one LED emits a wavelength within the ultraviolet and visible light range of electromagnetic spectrum; and
an LED control module adapted to receive instructions or data from a smart device and turn the at least one LED in the LED array on and off for predetermined periods of time based on the instructions received from the smart device.

2. The peripheral device of claim 1, wherein the LED array comprises a plurality of LEDs.

3. The peripheral device of claim 1, wherein the LED array emits a wavelength ranging from about 200 nm to about 800 nm.

4. The peripheral device of claim 3, wherein the LED array emits a wavelength selected from the group consisting of about 695 nm, about 635 nm, about 465 nm, and about 315 nm.

5. The peripheral device of claim 1, further comprising a first LED and a second LED, wherein the wavelength of the first LED and the second LED is different.

6. The peripheral device of claim 1, wherein the LED control module comprises a communication port adapted to physically connect with and transmit and receive data from a data port of a smart device.

7. The peripheral device of claim 1, wherein the LED control module comprises a wireless communication port adapted to wirelessly connect with and transmit and receive data with a smart device.

8. The peripheral device of claim 1, wherein the LED array is removable,

9. The peripheral device of claim 1, wherein the LED control module further comprises a rechargeable battery.

10. The peripheral device of claim 1, wherein the LED driver adjusts the intensity of the LED array based on instructions received from the smart device. cm 11. A method for treating a skin condition comprising the steps of:
providing at least one skin treatment protocol within an application on a smart device, the at least one skin treatment protocol providing a predetermined wavelength of light for a predetermined period of time;
connecting an LED based, skin treatment peripheral device to the smart device, wherein the LED based, skin treatment apparatus comprises:
a LED array comprising at least one LED, wherein the at least one LED emits a wavelength corresponding to the predetermined wavelength of light; and
an LED control module adapted to receive instructions from the smart device and turn the at least one LED in the LED array on and off for the predetermined periods of time based on the treatment protocol; and
selecting a skin treatment protocol from the at least one skin treatment protocol;
running the selected skin treatment protocol; and
exposing the skin of a patient to the LED based, skin treatment peripheral device during the step of running the selected skin treatment protocol.

12. The method of claim 1, further comprising the step of recording an image of the skin after treatment.

13. The method of claim 1, wherein the smart device is selected from the group consisting of a phone, watch, computer tablet, and portable computer.

14. The method of claim 1, wherein the treatment protocols are selected from the group consisting of skin ailments, wounds and pain.
